(19) United States
(12) Patent Application Publication
(10) Pub. No.: US 2011/0085135 A1
(43) Pub. Date: Apr. 14, 2011

(54) Free space hands free ocular observation camera mount

(76) Inventor: Eugene Robert Bertolli, Durham, CT (US)

(21) Appl. No.: 12/228,241

(22) Filed: Aug. 11, 2008

(51) Int. Cl. A61B 3/14; A61B 3/10

(52) U.S. Cl. 351/206; 351/245

(57) ABSTRACT

The present invention provides a stable mount for a small video camera to be donned by a (but not limited to) a human subject in order to view and record the subjects eyes and adnexa (the area around the eyes including the lids and surrounding tissues which the orbicularis muscles reside beneath), without disturbing the subject's side vision. The preferred embodiment uses a reduced spectacle like frame or may use a headset, which does not obscure the side vision of the subject, and contains a boom or arm which extends forward, and again without interfering with the subjects side vision. The invention additionally does not interfere with the examiner's view of the subject's eyes and adnexa. Additionally, the subject is free to move the body in space, and is free to move the head in various positions.

### BRIEF DESCRIPTION OF DRAWINGS

FIGS. 1 A and B shows an exploded view of the invention.

FIG. 2 A-E depicts viewing angles of the invention as worn by the subject, and during examination.

FIG. 3 depicts the invention worn by the subject in the head position for the Romberg test.

FIG. 4 A depicts the invention worn by a subject while having a “cover test” performed in conjunction with a “paddle” or occluder constructed of a material which blocks visible light, yet allows transmission of infrared light so as to allow imaging of the covered eye via the infrared capable camera mounted to the invention which otherwise would not be apparent, displayed in the monitor FIG. 4B.

FIGS. 5 A and B depicts 2 other embodiments of the invention.

FIGS. 6 A and B shows two views of one embodiment of the invention with an attached monitor/recorder unit.

FIGS. 7 A and B depicts the invention in two embodiments in conjunction with a visible light blocking visor which additionally allows transmission of infrared light so the eye activity may be observed and recorded by the infrared camera, 7C depicts the reverse side of the visor revealing the integral switched light stimulation.

FIG. 8 depicts the invention with a set of bubble levels which display the angle of the head of the subject relative to the ground, additionally with a millimeter rule as a reference for comparison of the iris pupil size, all of which are imaged via the camera.

FIG. 9 A depicts the invention with an additional camera mounted in a forward direction, along with a two channel receiver, splitter and monitor recorder, FIG. 9B.

FIG. 10 A-F Shows the invention as a component along with other components in a system of camera units.

FIG. 11A,B,C,D Shows components depicted in FIG. 10 as an example used in a system.

### DETAILED DESCRIPTION

The present invention is a free space/hands free day/night video/microphone headgear mounting system for observing and or recording eye movements, retinal reflex changes and iris pupil changes, allowing a visually non obstructed horizontal and vertical peripheral field of the subject, and full view of the subjects eyes by the examiner which is made up of the following required elements:

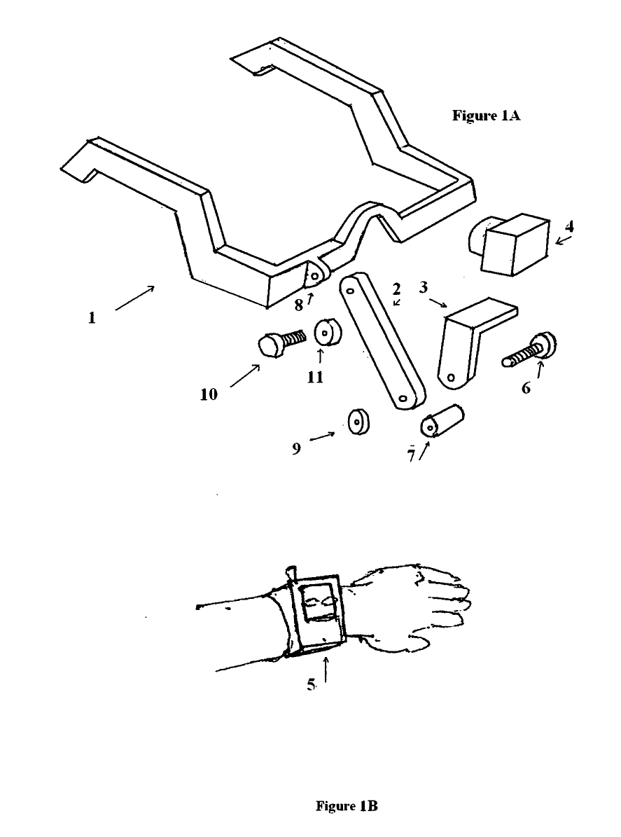

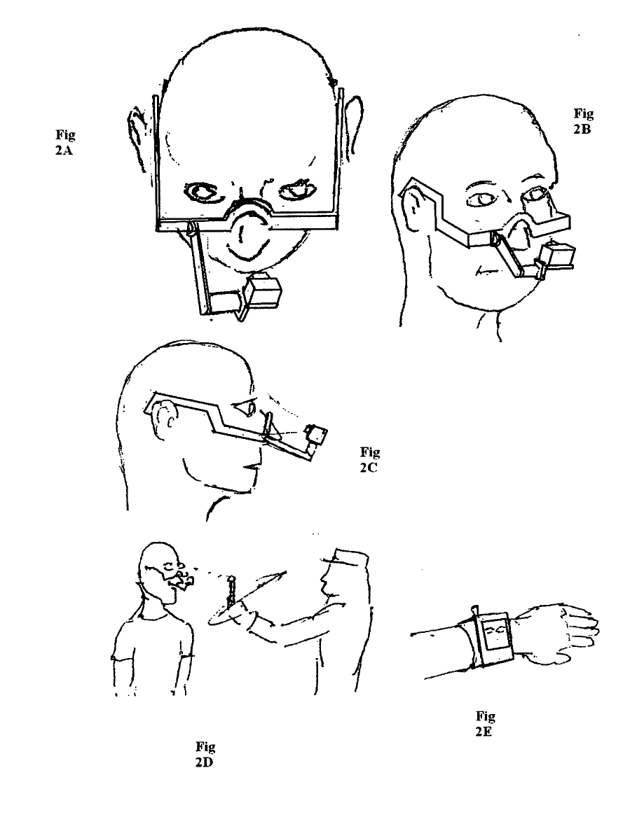

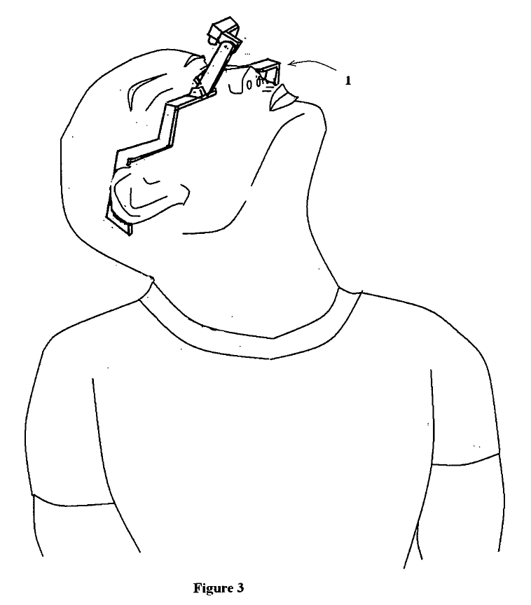

(FIGS. 1 A and B) A reduced spectacle like frame (1) and a boom (2) with a mount (3) for a day/night video camera with infrared illumination/microphone unit (4). This video camera with infrared illumination/microphone portion transmits motion pictures and sound to a receiver via radio wave or direct wire, where the imaging and sound is displayed and/or recorded on a (5) audio video monitor and/or to an audio/video recorder, and/or data storage device. These elements are connected as follows: the day/night video camera (4) with infrared illumination/microphone unit is attached to the mount (3) via adhesive, hook and loop, screw, bolt or other method. Hook and loop would provide a convenient attachment/detachment for ease of component exchange if necessary. The camera mount assembly (3) is fastened on one end of the boom (2), via, but not limited to rivet, screw, pivot, pin, bolt or other method: bolt (6) with spacer (7) and threaded knob (8) is shown, with the lens directed toward and viewing the subject's (wearer's) eyes. The boom/mount assembly (2,3,6,7,8,) holds the day/night video camera with infrared illumination/microphone mounting unit (4) medially toward the subject's eyes, in said manner to prevent peripheral field occlusion or restriction, and allowing examiner view. The boom (2) is fastened to the tab (9) on the front of the reduced spectacle frame (1) via rivet, pin, bolt, or other method; A bolt (10) with a threaded knob (11) is shown. One of the features that makes this apparatus unique, is that the assembly allows an unobstructed view of the subject's eyes by the examiner, and allows the subject to have unobstructed peripheral vision, from the boom angle set below the subject's eyes (FIG. 2 A overhead, 2B three quarter view, 2C side view, 2D view of subject wearing device while being tested and 2E is of component 5 wrist receiver/monitor/recorder unit). Further more, the subject is free to move such as in walking if eye movements, pupils, lid tremors, etc. are to be observed/recorded, or head position may be varied such as in the Romberg, where the eye head is angled back, eyes toward the sky, and closed, and where lid tremors are a finding of interest. (FIG. 3), or doll's head movements.

In one method, but not limited to, of testing the eye movements, the examiner moves a target to the extreme range of eye travel laterally, and also upwards, and also brings the target from approximately 1 to 2 feet out, then inward to the subject's nose, observing eye coordination, convergence of the eyes, and nystagmus if present. This apparatus allows the examiner to keep the hands free, and is only required to move the stimulus (target, such as a finger, pen, penlight etc.) for which the subject is to follow with his/her eyes. The 12-15 inch testing distance (as required for the Horizontal Gaze Nystagmus exam of the Standardized Field Sobriety Tests) is easily maintained with this apparatus. Pupil testing in absolute darkness is recorded and monitored; direct light stimulus may be directed at the subject's eyes to record and monitor the light pupil response; the subject's pupil response is also recorded in normal room light.

The apparatus may be used by, but not limited to, eye doctors, emergency room personnel, ambulance personnel, researchers, teachers, neurology, audiology for nystagmus and vestibular testing, distance medicine, law enforcement for testing the eyes for signs consistent with alcohol, substance or drugs which cause central nervous impairment, and recording the eye response (eye movement and iris pupil changes) for the examination (Driving Under the Influence enforcement—D.U.I.). This recording may be used for but not limited to, training and critiquing clinicians, students, law enforcement officers in the DUI examination of the eyes, or may be utilized for health care clinicians for recording eye movement and pupil responses, and for training healthcare professionals, in critiquing and refining the examination skills, and to demonstrate findings. The microphone conveys sound for recording and monitoring the examiner's instructions, and the subject's verbal response. In behavioral optometry and in sports vision training, the subjects eye performance may be recorded before training and then after training to demonstrate the level of improvement, such as the performance of saccades, pursuits, ocular alignment, anisocoria, strabismus, abnormal and normal and other measures and techniques.

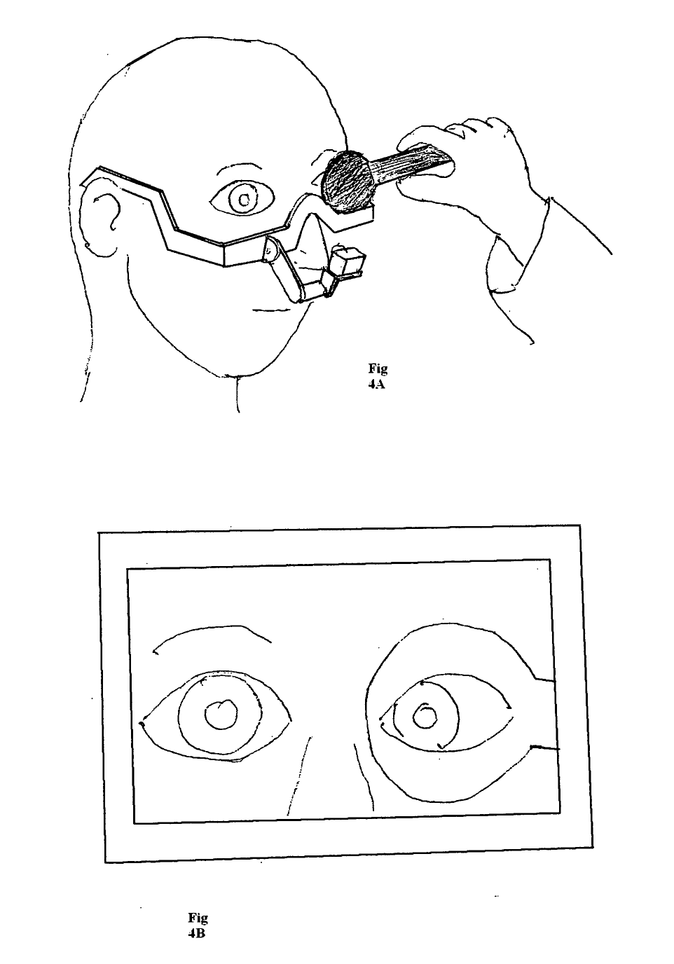

Other eye observation may be performed such as cover test, eye tracking, the introduction of prism lenses. With the benefit of being hands free, the examiner is capable of using various testing devices, for instance, but not limited to, lenses, filters, occluders, light stimulus, targets. A visible blocking material that allows infrared light to pass can be used as an occluder for the “cover, uncover test” utilized by eye doctors. The material blocks the visible light which is necessary to perform the test, so what the covered eye is doing behind the occluder is not seen by the examiner with the naked eye (FIG. 4A), but is observable from the infrared equipped video camera images on the monitor in real time or for later analysis with the recorded material (FIG. 4B).

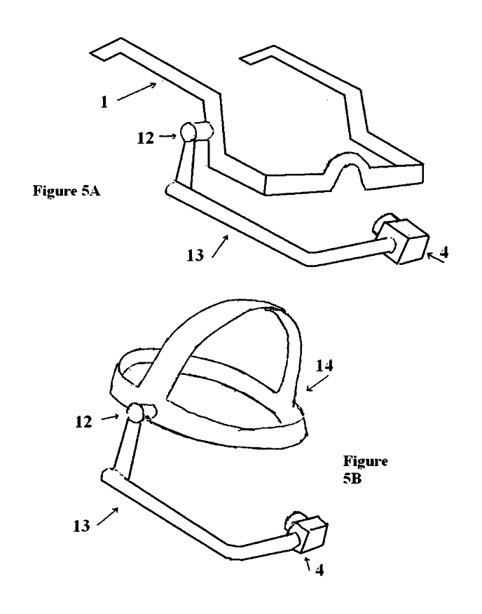

The invention may be worn via a reduced spectacle like frame (FIG. 1) (FIG. 2), (FIG. 5 A) or with a head band or headgear (FIG. 5 B). The boom (FIG. 5 A component 13) may be attached to the side of the frame (FIG. 5 A component 1) via a pivot (component 12), the front of the frame as in FIG. 1, or the side of the headset (FIG. 5 B component 14) and is set low, so as not to obstruct viewing by the day/night video camera/microphone unit (FIG. 5 component 4), and allows unobstructed peripheral view of the subject by the examiner. An adjustable pivot (FIGS. 5A and 5B component 12) allows the examiner to make boom (FIGS. 5A and 5B component 13) angle changes if necessary for the day/night video camera view.

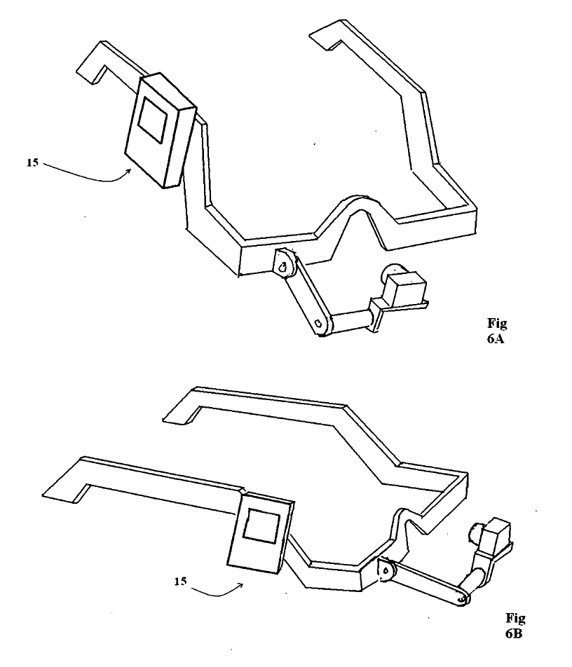

The apparatus may be constructed of many materials such as plastic or metal, but light weight rigid, and durable plastic is preferred. In testing the eyes, it is preferable to use a visible light capable/Infrared capable video element, and an integral IR light source, plus integral microphone, allowing monitoring, recording and further analysis, of the but not limited to the subject. Other cameras with various capabilities may be mounted at the pleasure of the user. Many small light weight video cameras with combinations of wireless feature, multiple channels, self data storage, various lighting sources and wavelength capabilities are available or possible, with various applications. One of the configurations that suit a wide variety of testing conditions and collection of information is the day/night video camera/microphone unit (4) which is capable of acquiring images in lighted conditions, and capable of acquiring images in dark conditions with preferable integral infrared illumination which is invisible to the human eye, yet visible to the video camera. The day/night video camera/microphone unit (FIG. 1A component 4, FIGS. 6A and 6B component 4) may use wires to transmit the image/sound to a video/audio recording unit or other data storage device or to a monitor/recorder mounted on the apparatus (FIG. 6A and B component 15), or preferable use wireless signal transmission such as, but not limited to, radio frequency to a radiofrequency receiver (FIG. 1B component 5) combination monitor recording unit.

The simplest version of the apparatus is the day/night video camera/microphone unit (FIG. 1A), attached to the boom assembly, which in turn is attached to the spectacle like frame worn by the subject. (Two video camera assemblies may be used one for each eye, with its own transmission channel, yet requiring a signal splitter, on the receiver unit, but one camera is sufficient to view both eyes simultaneously, reducing weight of the unit, and maintaining a more efficient unit. A camera assembly is directed to the eyes)

Direct wiring harness to an audio/video recorder with monitor and speaker for use by the examiner conveys and records the image and sound, or preferably but not limited to, transmitted via radio wave, or infrared beam, to a receiver to a data storage, monitor, recording device, or if desired, a combination of such, or a self contained data storage device.

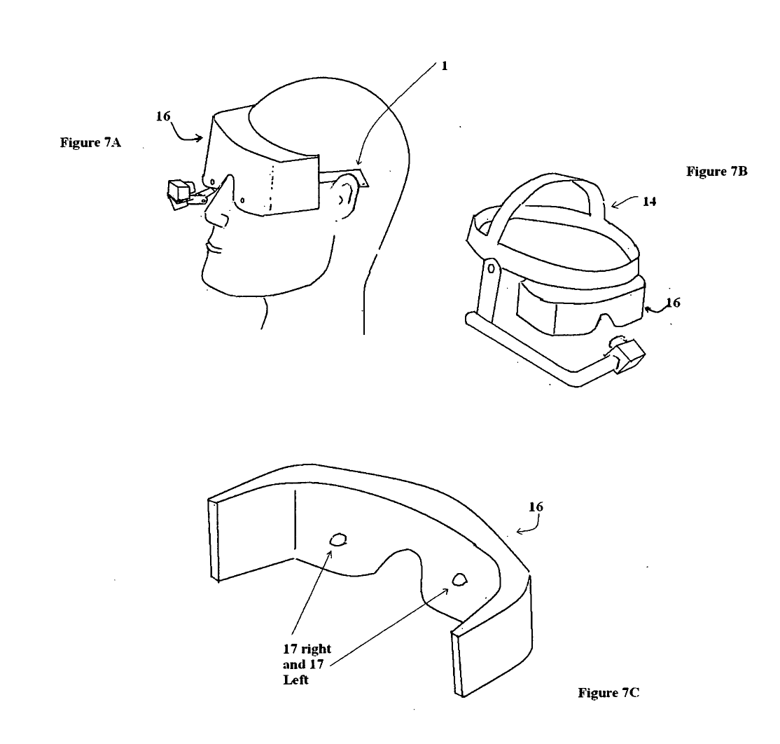

The deluxe version of the apparatus would be preferably, but not limited to, a reduced spectacle frame or a headgear, with an adjustable pivot for the video/microphone holding boom. Wireless (radio-wave for example) transmission of the image and sound is transmitted to a wireless audio/video receiver, which inputs into a recorder/monitor/speaker. An additional goggle (FIG. 7 A, 7 B, 7 C component 16) allowing the transmission of infrared light only, with no or minimal visible light, is worn by the subject, while also wearing the headset (FIG. 1, FIG. 5A, or FIG. 5B) shown in combination in (FIG. 7A 7B), for simulated dark room conditions. The day/night video camera with infrared illumination is capable to view the eyes through the infrared light only, or very dark goggles. The goggle (FIG. 7 component 16) is worn for testing the pupil size under dark conditions; this is an important finding for refractive surgeons that the size of the pupil in dark conditions does not exceed the anticipated surgical optical zone. An internal light source (FIG. 7C, components 17 right and 17 left) in the goggle for each eye individually may be activated by a switching mechanism, by the examiner, or examiner's assistant if applicable, to observe the direct light stimulus by the infrared video camera.

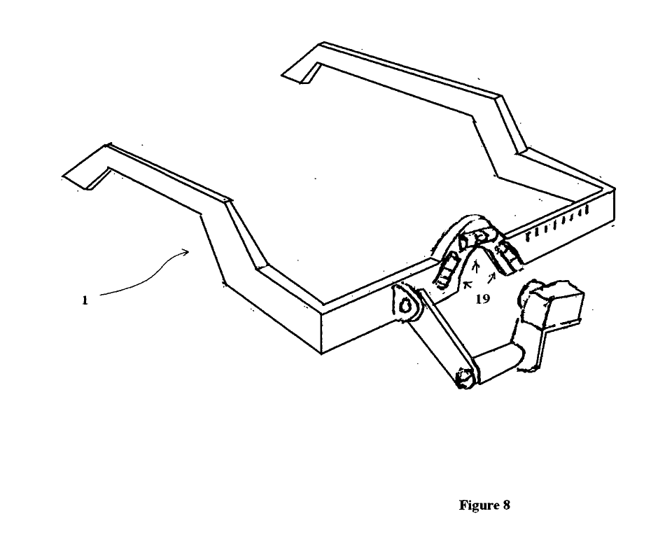

The goggles are moved out of position by the examiner for recording and observing the pupil size in normal room illumination. A permanent (FIG. 8 component 18) (or a temporary) millimeter scale may be used with the apparatus to use as a guide to document and/or train the examiner in estimating iris pupil size. A temporary placed or permanent millimeter scale is useful for training the examiner(s) in estimating iris pupil sizes and making a record of the pupil sizes. This millimeter scale may be, but not limited to be incised, imprinted or attached to the reduced spectacle frame front surface, or a (preferably stiff paper), plastic millimeter scale placed on the nose of the subject wearing the headset.

A bubble or other level may be mounted to the front of the spectacle like frame in view of the camera allowing head angle position for instance when vestibular input is of interest, such as tilting the head 45 degrees toward one shoulder to view the presence of, but not limited to positional alcohol nystagmus (FIG. 8 components 19 right, left and center).

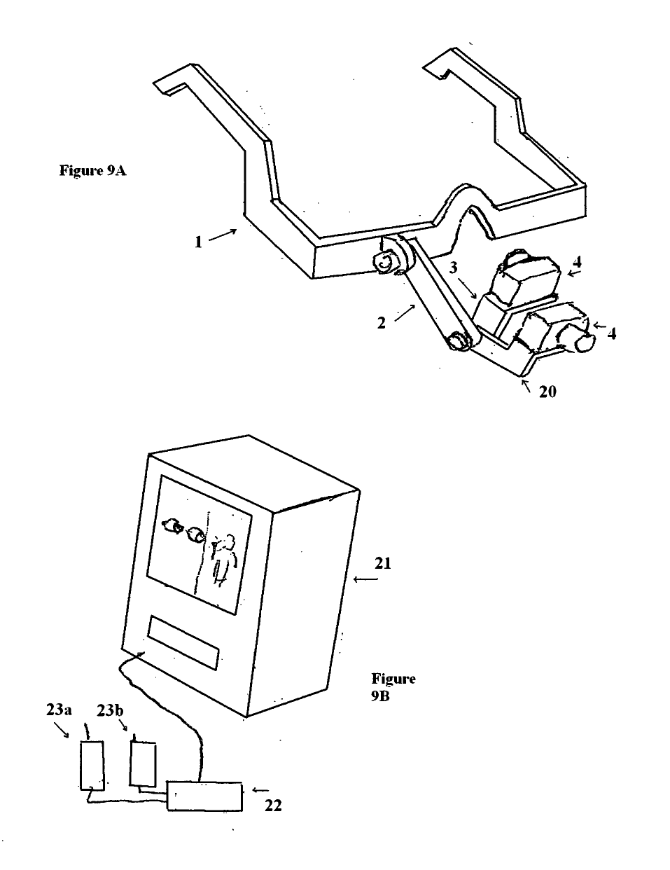

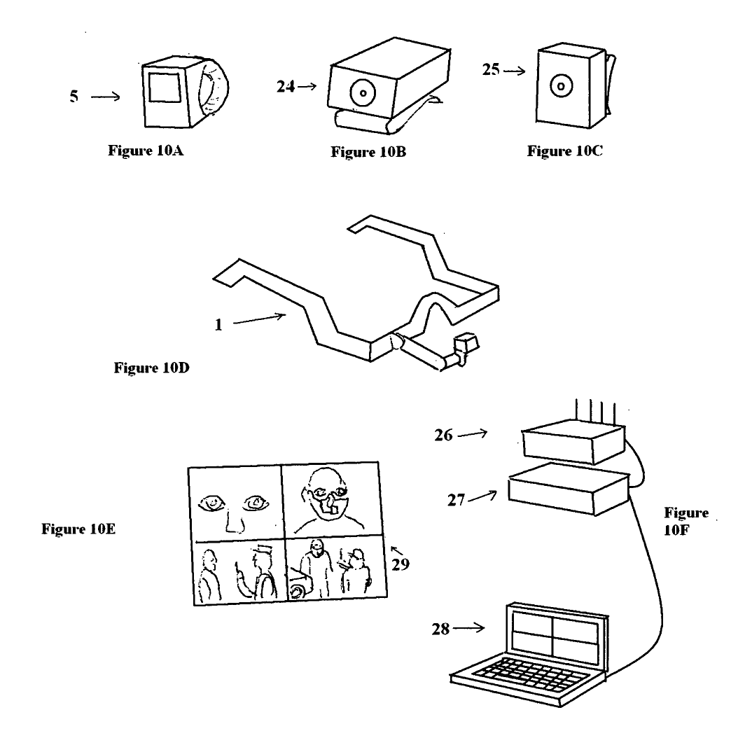

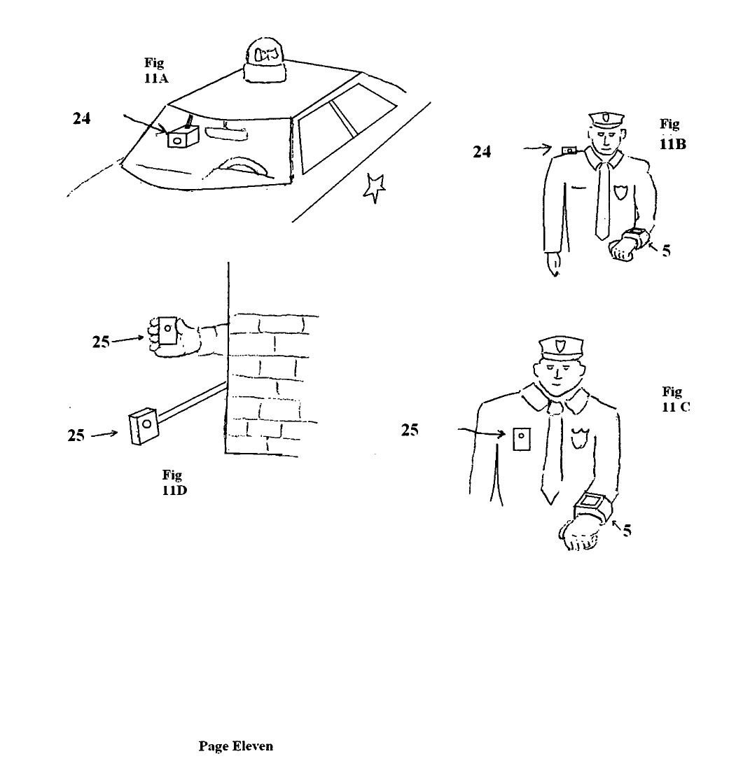

A multiple camera system, while wired system is possible, wireless is desired allowing freedom of movement. Each camera has its own channel, and receiver. The receiver for each camera may feed its own monitor/recorder and/or may feed into a splitter where all the cameras may be monitored and or recorded, or one camera input may be selected. The cameras may be mounted on the headset, or may be externally placed, for instance for a long view of the subject, the subject and the examiner, the examiner or what ever scene is required. Two or more camera units may be mounted on the headset. For instance, but not limited to, one camera unit toward the subject's eyes (FIG. 9A component 4), the other toward the test stimulus (FIG. 9A second component 4 attached to mount component 20), whether a moving light source, reading material, etc. The receivers (FIG. 9B components 23a and 23b) each set at separate and corresponding channels to the cameras (4) input the individual signals into a splitter (FIG. 9B component 22), and then into the monitor and or recorder (FIG. 9B component 21). One application is analyzing the eye movements to the moving stimulus(i) either by human or computer analysis. This would show, but not limited to, the quality of the tracking ability, or the speed of tracking etc. The “forward” facing camera may also be utilized to observe and critique the examiner. Other systems may be the headset camera assembly (FIG. 10D headset 1) on one channel, a dash camera (FIG. 11A with component 24 mounted on visor or post) on another, an epaulet (FIG. 10B component 24) or shirt camera (FIG. 10C component 25) on one officer, or both, all with their own channels. A multi channel receiver (FIG. 1OF component 26) may be mounted in the vehicle, which may input a splitter (FIG. 1OF component 27) which in turn inputs a monitor, or recorder, or laptop (FIG. 1OF component 28, image display representation FIG. 10E #29 viewing the image of the headset, image of the officer's view, image of the dash view, and image from the second officers view, clockwise from upper left), and or transmit the images to the precinct or other station as desired. Each officer may be equipped with a wrist mounted or other format of multichannel receiver/monitor/recorder (FIG. 10A component 5). The wrist unit (5) may be used with the epaulet (FIG. 10B component 24) (FIG. 11B) or shirt mounted (FIG. 10C component 25) (FIG. 11C) camera as an auxiliary night vision system and or may be temporarily mounted but not limited to a night stick (FIG. 11D) for looking around corners or under a vehicle etc.

I claim:

1. a free space ocular observation subject donned camera mount comprising a spectacle like frame which does not occlude or restrict the subject's peripheral vision

2. the free space ocular observation camera mount in claim 1, wherein said at least one boom originating from the front or side of the spectacle like frame, configured in such a manner as to hold the camera, yet not interfere with the subject's peripheral vision

3. the said invention of claims 1 and 2 allows the camera to be continually and with stability viewing and recording the subject's ocular findings foe various purposes, under various test conditions with freedom of movement of the subject's head and body method for ocular finding(s) recording, observation and or monitoring:

4. the said claims of 1, 2, 3 may be incorporated into a system of many observation cameras, not limited to, providing multiple views of the subject, subject's ocular findings, target of regard, examiner, etc. which may be used for various analysis, human and or computer assisted.

5. The said claims of 1, 2, 3, 4, by utilizing a near infrared capable camera allows viewing and recording the subject's eyes, adnexa, and test responses in total darkness.

6. The said claims 1, 2, 3, 4, 5, with an eye occluder which blocks visible light from the subject's eye, yet allows transparency to the near infrared camera, provides observation and recording of the occluded eye's behavior which otherwise would be obscured to the examiner.
